(19) United States
(12) Patent Application Publication
(10) Pub. No.: US 2025/0366808 A1
(43) Pub. Date: Dec. 4, 2025

(54) Device for periapical radiography

(71) Applicants: Angel Manuel Eyaralar Argüelles, Gijon Asturias (ES); Angela Eyaralar Alvarez, Gijon Asturias (ES); Cristina Eyaralar Alvarez, Gijon Asturias (ES)

(72) Inventors: Angel Manuel Eyaralar Argüelles, Gijon Asturias (ES); Angela Eyaralar Alvarez, Gijon Asturias (ES); Cristina Eyaralar Alvarez, Gijon Asturias (ES)

(21) Appl. No.: 19/105,384

(22) PCT Filed: Aug. 18, 2023
(86) PCT No.: PCT/ES2023/070520

§ 371 Date: Feb. 21, 2025

(30) Foreign Application Priority Data
Aug. 21, 2022 (ES) P202230760

(51) Int. Cl. A61B 6/51; A61B 6/00; A61C 1/08

(52) U.S. Cl. CPC A61B 6/512; A61B 6/52; A61C 1/084

(57) ABSTRACT

The present disclosure relates to a device for periapical radiography that enhances accuracy in dental implantology. It includes a connection element that secures a radiographic plate holder relative to an anchoring element, such as a direction indicator, depth gauge, or drill. The device ensures parallel alignment with the radiographic plate and perpendicularity to the X-ray beam, minimizing distortion. This allows precise intraoperative periapical radiography, reducing reliance on CBCT or panoramic imaging. Configurations include an L-shaped bar, a tube or sleeve, and a fixation base, all designed for stable alignment. Compatible with standard implant systems, the device offers a cost-effective, accurate, and repeatable method for real-time implant verification. Additionally, it enhances procedural efficiency, improves patient safety, and reduces radiation exposure.

### FIELD OF DISCLOSURE

The present disclosure relates to a device for periapical radiography in dental implantology. More particularly, it concerns a radiographic device designed to enhance the accuracy and reliability of intraoperative periapical radiographs used during dental implant placement.

### BACKGROUND OF THE DISCLOSURE

Dental implantology has become a widely accepted solution for the rehabilitation of partially or completely edentulous patients. The success rate of dental implants is high in the short, medium, and long term, effectively restoring both masticatory function and aesthetics. This has significantly improved the oral health and quality of life of millions of patients worldwide. Currently, more than 10 million dental implants are placed annually, and the demand continues to grow.

Proper implant placement is crucial to achieving long-term success. From both functional and aesthetic perspectives, implants must be positioned with high precision. The ideal implant should be as long as possible while maintaining an optimal spatial orientation as determined by the chosen surgical technique. However, placement is constrained by the available bone volume and the presence of adjacent anatomical structures that must be preserved, including:
The nasal floor
The maxillary sinus floor.
The inferior alveolar nerve.
The incisal nerve
The pterygomaxillary fossa
The palatine nerve.
The roots of neighboring teeth

To assess bone volume and locate these anatomical structures, radiographic studies are performed during pre-surgical planning. The most common imaging techniques include:
Periapical radiographs
Panoramic radiographs
Computed Tomography (CT scans).
Cone Beam Computed Tomography (CBCT)

Despite these preoperative imaging techniques, discrepancies often arise when transferring pre-surgical radiographic information to intraoperative conditions. Errors in drilling angulation and depth can occur, increasing the risk of damaging critical anatomical structures. Therefore, a method for real-time verification of drilling accuracy intraoperatively would be highly beneficial. Such a system would allow clinicians to:
Confirm the correct drilling direction
Ensure the drilling depth respects anatomical boundaries
Make real-time corrections before implant placement

One of the most widely used radiographic techniques is the parallelism or long-cone radiographic technique, introduced by Dr. Fitzgerald. This method minimizes geometric distortion, producing radiographs that are accurate in size and shape. However, several key requirements must be met:
The object (implant site) and the radiographic plate must be parallel.
The central X-ray beam must be perpendicular to both the object and the plate.
The radiation source must be at a minimum distance of 40 cm (double that of other periapical techniques), reducing projection distortion

If these conditions are not met, image distortion occurs, leading to measurement errors that compromise the accuracy of implant placement.

To facilitate periapical radiography, film holder systems are required to maintain:
The parallel positioning of the radiographic plate relative to the object.
The perpendicular alignment of the X-ray beam

Since 1968, the Rinn device has been the standard for achieving these conditions. The Rinn device consists of:
A plate holder with a bite block
An arm connected to a centering ring, which aligns the radiation beam

This system ensures that the X-ray beam remains perpendicular to the radiographic plate while keeping the object and plate parallel. It is currently used in almost all dental clinics worldwide for obtaining high-quality periapical radiographs.

However, the Rinn device was originally designed for intraoral radiographs of teeth and has remained largely unchanged since its introduction. It does not address the specific challenges posed by dental implantology.

### DETAILED DESCRIPTION OF THE DISCLOSURE

### Existing Challenges in Periapical Radiography for Implantology

The long-cone periapical radiographic technique provides high accuracy when performed correctly. However, applying this technique in implantology presents major limitations, as there is no fixed reference point to align the radiographic plate with the implant-related instrument (e.g., direction indicator, depth gauge, drill). The lack of precise alignment leads to geometric distortion, making it difficult to obtain reliable measurements.

Today, the use of endosseous implants has become the most efficient technique for replacing lost teeth.

In 1992, Dr. Gelb developed and patented radiographic depth gauges (Gelb D A. Gelb Depth Gauge: A diagnostic aid in implant placement. Int J Periodontics Restorative Dent, 1992, 12(4):300-309). These are longitudinal devices initially manufactured in two diameters, 2 mm and 2.3 mm, and in two lengths, 13 mm and 20 mm. These devices have constrictions at predefined lengths of 8.5, 10, 11.5, 13, 15, 18, and 20 mm.

The purpose of these guides, probes, or depth gauges is as follows: once the probe is inserted into the osteotomy made with a drill of the same diameter as the guide, a periapical radiograph is taken to determine the distance to neighboring anatomical structures (maxillary sinus, nasal fossa, inferior alveolar nerve), as well as the parallelism with adjacent teeth or other implants.

The problem is that it is practically impossible to achieve and maintain parallelism between these Gelb guides and the radiographic plate, making the plates highly unreliable, as reported by the research group at New York University.

The issue becomes more severe when the object being radiographed is no longer a tooth but rather an element used in oral implantology, such as a direction indicator, a depth gauge, or a drill.

The depth gauges developed by Dr. Gelb in 1992 were designed to allow the surgeon to verify intraoperatively, with precision, whether the drilling of the implant site is correct in terms of both angulation and depth, and, if necessary, to adjust or complete it properly. To achieve this, metallic guides of 2 mm in diameter with markings at various lengths corresponding to implant sizes (7, 10, 13, 15, 18, and 20 mm) are used. A Rinn-type plate holder was utilized to ensure the X-ray beam was perpendicular to the plate.

Currently, nearly all implant systems available on the market have incorporated depth gauges similar to those developed by Dr. Gelb into their surgical kits.

The problem, as previously mentioned, is that it is very difficult, if not impossible, to ensure that the object being radiographed (Gelb's depth gauge) is parallel to the plate and perpendicular to the X-ray beam, and therefore, to obtain an accurate radiograph.

In 1995, Dr. Gher published a study comparing the accuracy of different radiographic techniques used during the placement of dental implants (Gher M E, Richardson A C. The accuracy of dental radiographic techniques used for evaluation of implant fixture placement. Int J Periodontics Restorative Dent, 1995:15(3): 268-283).

His study compared periapical radiography, panoramic radiography, linear tomography, and computed tomography. The study was not conducted on live patients but rather on a specimen of a human hemimandible. The study concluded that periapical radiography, when the X-ray beam is perpendicular to the plate and the object, provides the most accurate measurement with the least variation among all evaluated techniques. The measured lengths varied between 0.0 and 0.3 mm compared to actual dimensions. When the beam angle shifted to 80, 70, and 60 degrees, the margin of error increased.

The study also concluded that the precision obtained is likely higher than what can be achieved under real clinical conditions, as the mandible in this case was completely stable during the study.

In an article published by Dr. Kakumoto from New York University on the accuracy of periapical radiographs, the results showed that the measurement failed in 66% of cases, with an error range extending from −1.69 mm to +2.1 mm (Kakumoto T, Barsoum A, Froum S J: Accuracy of cone-beam computed tomography versus periapical radiography measurements when planning placement of implants in the posterior maxilla: A retrospective study. Compend Contin Educ Dent. 2021-July-42(7):e1-e4).

The New York University article concluded that the long-cone technique is not applicable in oral implantology because there are no key reference points, such as the incisal edge, making measurement difficult and introducing errors, as the surgeon cannot achieve precise parallelism between the plate and the measuring instrument. The article also concluded that the accuracy obtained by Dr. Gher in his cadaver study may be considered difficult, if not impossible, to replicate in live patients.

A well-known technique in this field is computer-guided surgery. In this technique, data from CT or CBCT scans is used to generate a DICOM file, which can then be used to print a stereolithographic replica of the patient's bone structure. These resin models are used to create surgical guides, which enhance the accuracy and precision of implant placement. However, even these sophisticated, expensive, and relatively inaccessible systems can introduce errors.

In a study conducted by Dr. M. Yeung and collaborators from Virginia Commonwealth University (Accuracy and precision of 3D-printed implant surgical guides with different implant systems: An in vitro study. J Prosthet Dent 2020; 123:821-8), the researchers warned that in guided surgery, special attention must be paid to the vertical depth of implant placement. The depth of the osteotomy should be confirmed before placing the implant, and the vertical placement length must be considered, as errors of approximately 3 mm or more may occur. Surgeons should also be cautious of potential vertical and palatal displacement.

Given the above, the current state of the art indicates that:
1. Periapical radiographs are highly precise.
2. To achieve this precision, the parameters established in the long-cone technique (introduced by Dr. Fitzgerald) must be followed.
3. The ideal scenario is practically impossible to achieve in real clinical conditions.

Considering the current challenges in the field, it would be desirable to develop a system that allows intraoperative radiographs of depth gauges, direction indicators, or drills using the long-cone technique.

The present disclosure provides a solution to these problems.

The Gelb depth gauges, developed in 1992, were designed to allow the surgeon to verify implant bed preparation intraoperatively by providing a radiographic reference. However, their use has significant limitations:
Parallelism cannot be maintained between the depth gauge and the radiographic plate.
Image distortion occurs, leading to incorrect depth and angulation measurements.
Implant misalignment and anatomical damage may result from inaccurate radiographs.

Studies conducted at New York University have demonstrated that periapical radiography, when not performed with perfect alignment, has error margins of up to ±2 mm, which can significantly affect the precision of implant placement.

Given these limitations, a novel device is required to rigidly align the implant-related elements with the radiographic plate and X-ray beam, ensuring an accurate periapical radiograph.

### Solution Provided by the Present Disclosure

The present disclosure provides a device for periapical radiography that eliminates geometric distortion by maintaining:
A rigid parallel relationship between the radiographic plate and the implant-related instrument.
A fixed perpendicular alignment between the X-ray beam and the radiographic elements.
A secure intraoperative reference for verifying implant position and depth.

The disclosed device comprises:
A connection element (1) that links the plate holder (3) to an anchoring element (2) (direction indicator, depth gauge, or drill).
A plate holder portion (13) that ensures the radiographic plate remains in a fixed position.
An anchoring portion (12) that securely connects to the depth gauge, direction indicator, or drill.

This configuration enables the surgeon to obtain real-time intraoperative periapical radiographs with minimal distortion, improving implant placement accuracy.

### Configuration of the Disclosed Device

The disclosed device can be implemented in three different configurations, depending on the radiographic setup:

### 1. Rinn Plate Holder-Based System

In this configuration, the disclosed device is rigidly attached to a Rinn plate holder and an anchoring element. The anchoring element can be:
A direction indicator (with or without depth markers)
A depth gauge.
A drill (with calibrated lengths or adjustable stops)

This system ensures that:
The radiographic plate and implant-related element remain parallel.
The X-ray beam remains perpendicular to both, minimizing measurement errors.
   2. Integrated Plate Holder with Guiding Components

In this configuration, the disclosed device is incorporated into the horizontal portion of the plate holder's bite block in the form of:
A tube or sleeve that allows the insertion of depth gauges, direction indicators, or drills.
A guide mechanism that secures the implant-related element in the correct spatial orientation.

This system eliminates the need for external stabilization of the implant-related element by integrating it directly into the plate holder.

3. Modified Bite Block with Anchoring Mechanism

In this configuration, the bite block of the plate holder is modified to include:
Pre-drilled holes for inserting metal arms.
A base attachment that allows the depth gauge or drill to be securely positioned.

This anchoring system ensures that the implant-related element remains:
Securely positioned throughout the radiographic procedure.
Perfectly aligned with the radiographic plate and X-ray beam.

### Advantages of the Disclosed Device

The disclosed device overcomes the limitations of existing radiographic techniques and offers multiple advantages:

1. Compatibility with Existing Dental Radiography Equipment
Works with conventional periapical radiography systems.
Compatible with Rinn plate holders and commonly used depth gauges and drills.

### 2. Enhanced Radiographic Accuracy

Ensures parallel alignment between the implant-related instrument and the radiographic plate.
Prevents image distortion, leading to high-precision measurements.

### 3. Real-Time Verification of Implant Placement

Enables intraoperative radiographs to confirm implant angulation and depth.
Allows immediate corrections, preventing complications.

### 4. Safety and Reliability

Prevents displacement of implant-related elements during radiography.
Reduces the risk of aspiration or swallowing of depth gauges.

### 5. Cost-Effective Solution

Uses low-radiation periapical radiographs instead of expensive CT scans.
Eliminates the need for additional imaging techniques.

### 6. Minimization of Additional Imaging Needs

By improving periapical radiographic accuracy, the device reduces the need for CBCT and panoramic radiographs.

### BRIEF DESCRIPTION OF THE FIGURES

To further illustrate the disclosed device, a set of figures accompanies this description, which are presented for explanatory and non-limiting purposes:

FIG. 1: First system—the L-shaped bar has not yet been inserted into the plate holder, and the depth gauge is not yet attached.

FIG. 2: First system—the L-shaped bar is inserted into the plate holder, and the depth gauge is secured in place.

FIG. 3: Second system—integrated plate holder with guiding sleeve.

FIG. 4: First system—assembled device for periapical radiography.

FIG. 5: L-shaped bar with a short arm and a long arm at 90° for securing a depth gauge.

FIG. 6: L-shaped bar with a short arm and a long arm at 90° for securing a drill.

FIG. 7: Incorrect positioning of the radiographic elements, leading to a distorted image.

FIG. 8: Correct positioning of the radiographic elements, resulting in an accurate radiographic image.

FIG. 9: Depth gauges with varying diameters and lengths.

FIGS. 10-11: Third system—modified bite block with anchoring mechanism.

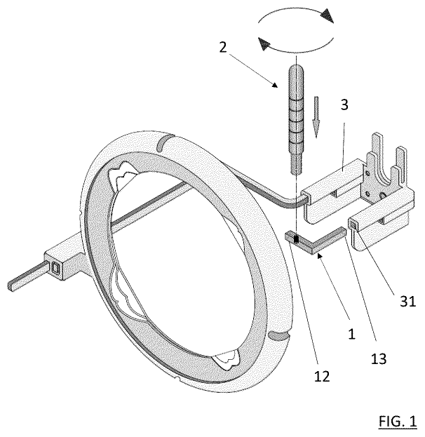

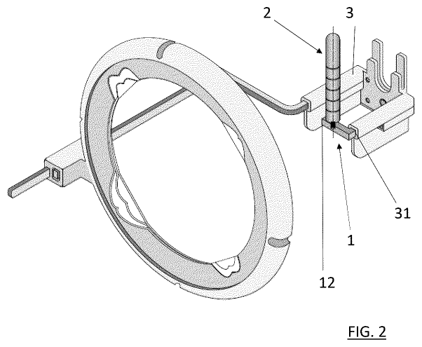

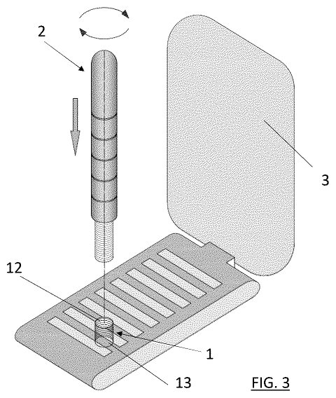

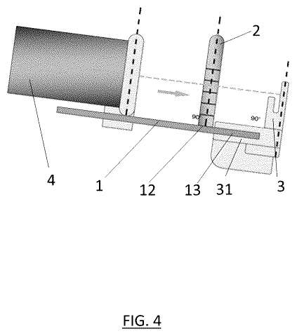

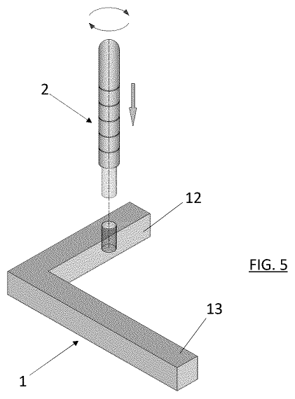

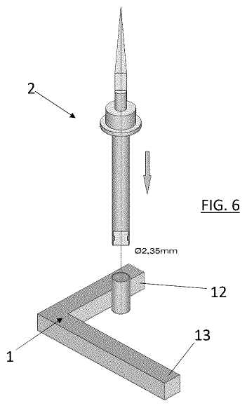

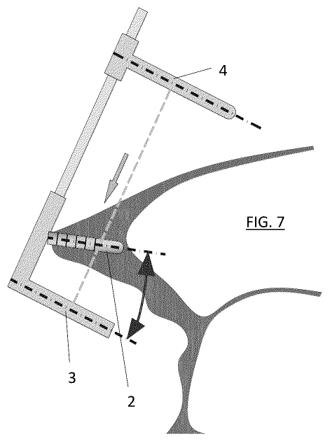

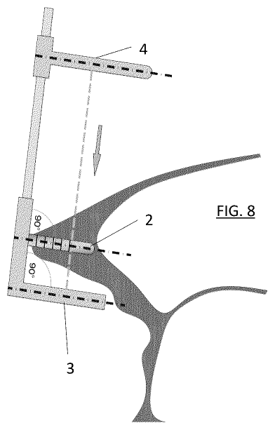

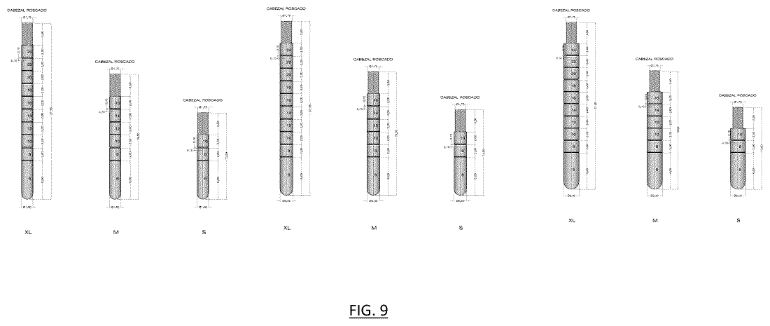

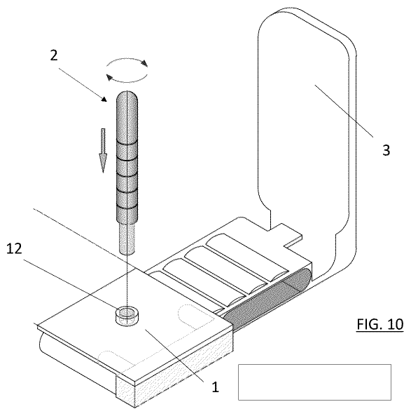

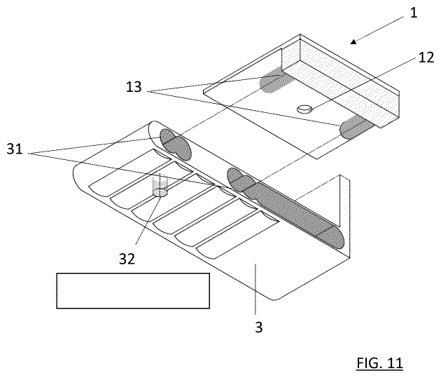

### DETAILED DESCRIPTION OF THE INVENTION

The disclosed device consists of a connection element (1) that establishes a rigid connection between:
An anchoring element (2) (direction indicator, depth gauge, or drill).
A plate holder (3) (holding the radiographic film).

The connection element (1) comprises:
An anchoring portion (12): Connects to the implant-related element (depth gauge, drill, etc.).
A plate holder portion (13): Inserts into the plate holder to maintain fixed alignment.

This structure ensures that the positioning of the radiographic plate remains unchanged relative to the X-ray beam, eliminating measurement errors.

### 1. L-Shaped Configuration

In some embodiments, the connection element (1) adopts an L-shape, where:
The plate holder portion (13) is positioned at one end.
The anchoring portion (12) is positioned at the other end.

This configuration provides:
Increased stability during radiography.
Rigid alignment between the implant-related element and the radiographic plate.

### 2. Sleeve Configuration

In another embodiment, the connection element (1) is a sleeve that:
Fits into the plate holder cavity.
Allows insertion of depth gauges, drills, or direction indicators.

This configuration simplifies component integration within the radiographic system.

### 3. Base Configuration

In some embodiments, the connection element (1) is designed as a base with:
Protrusions that align with cavities in the plate holder.
Fixation mechanisms that allow depth gauges or drills to be securely attached.

This provides:
Increased positioning accuracy.
Enhanced device stability during radiography.

The invention claimed is:

1. A device for periapical and intraoral radiography, comprising a connection element configured to connect a plate holder to an anchoring element, wherein the anchoring element is selected from a direction indicator, a depth gauge, or a drill, and is configured to be inserted into a patient's bone, the plate holder is configured to hold a radiographic film or sensor to obtain a periapical or intraoral radiograph, the connection element maintains the position of the plate holder relative to the anchoring element in a fixed arrangement, ensuring that both remain parallel to each other and perpendicular to an X-ray beam, and the device is compatible with different periapical radiographic techniques, including but not limited to the long-cone technique and the parallelism technique.

2. A method for obtaining an accurate periapical radiograph during a dental implant procedure, comprising placing a radiographic plate holder in a fixed position relative to an anchoring element selected from a direction indicator, a depth gauge, or a drill, ensuring the radiographic plate holder remains parallel to the anchoring element and perpendicular to an X-ray beam, and using the device to stabilize the positioning and eliminate image distortion during periapical radiography.

3. A device according to claim 1, wherein the device is used in dental procedures requiring periapical or intraoral radiographs, including but not limited to implantology, endodontics, periodontics, and orthodontics.

4. The device of claim 1, wherein the connection element comprises an anchoring portion and a plate holder portion, the anchoring portion being configured to attach to the anchoring element through a threaded or non-threaded connection, allowing compatibility with depth gauges of different diameters.

5. The device of claim 4, wherein the anchoring portion comprises a locking mechanism that prevents unintended movement during radiographic imaging.

6. The device of claim 5, wherein the anchoring portion is interchangeable to allow compatibility with multiple implant systems.

7. The device of claim 1, wherein the connection element has an L-shape, comprising a first end forming the plate holder portion and a second end forming the anchoring portion, which is configured to be attached to the anchoring element.

8. The device of claim 7, wherein the L-shaped connection element is a square-section bar of 3×3 mm, with a short arm of 18 mm and a long arm of 25 mm at a 90-degree angle.

9. The device of claim 1, wherein the connection element is a sleeve incorporated into a horizontal section of the plate holder's bite block, allowing insertion of the anchoring element.

10. The device of claim 9, wherein the sleeve has an internal diameter of 2.35 mm, allowing insertion of a standard drill shaft used in oral implantology.

11. The device of claim 1, wherein the plate holder's bite block comprises holes designed to receive metallic arms that connect to a centering ring.

12. The device of claim 11, wherein the holes accommodate a base that allows depth gauges or drills to be securely fastened.

13. The device of claim 1, wherein the anchoring element is a depth gauge that includes markings corresponding to standard implant lengths.

14. The device of claim 13, wherein the depth gauge is made of a radiopaque material, selected from surgical-grade stainless steel or titanium, allowing sterilization in an autoclave.

15. The device of claim 1, wherein the plate holder is a Rinn-type plate holder used in periapical radiography.

16. The device of claim 1, wherein the device allows intraoperative radiographs with high measurement accuracy.

17. The device of claim 1, wherein the device ensures proper alignment for periapical radiographs, reducing geometric distortion.

18. The device of claim 1, wherein the connection element is detachable, allowing replacement with different configurations according to the radiographic technique used.

19. The device of claim 1, wherein the depth gauge includes pre-defined length constrictions to aid in measuring the distance to anatomical structures.

20. The device of claim 1, wherein the anchoring element prevents displacement during the radiographic procedure, ensuring reliable imaging.
